(19) United States
(12) Patent Application Publication
(10) Pub. No.: US 2025/0304903 A1
(43) Pub. Date: Oct. 2, 2025

(54) METHOD FOR PREPARING QUALITY ASSURANCE AND QUALITY CONTROL REPORT ON MICROBIAL PRODUCTION PROCESS

(71) Applicant: SYSMEX CORPORATION, Kobe-shi (JP)

(72) Inventor: Joseph Rezell, Lincolnshire, IL (US)

(21) Appl. No.: 18/622,159

(22) Filed: Mar. 29, 2024

(51) Int. Cl. C12M 1/36

(52) U.S. Cl. CPC C12M 41/48

(57) ABSTRACT

This invention is to provide rapid microbiological test reports, using microbiological testing devices, such as fluorescent flow cytometry (FCM), fluorescent microscopy, and polymerase chain reaction (PCR), HPC, etc., to create a true and comprehensive microbiological description of each step of microbial production process in real-time. These microbiological descriptions are combined to create a unique baseline microbiological description or “Fingerprint” of the entire process.

### BACKGROUND

### Technical Field

This invention relates to a method for preparing a quality assurance and quality control report on microbial production process and more specifically to a method for providing a comprehensive analysis report on microbial activities observed throughout a microbial production process.

### Background Art

A microbial production process is generally understood to be the controlled use of microorganisms in the manufacture of products e.g., fermented foods and beverages, and in the treatment of wastewater. A widely-known exemplary application of a microorganism is the use of yeast to manufacture alcoholic beverages, such as wine, beer, etc. Especially, winemaking has now become a truly global enterprise significantly affecting the economic well-being of many countries.

Like other non-microbial productions, microbial productions, such as winemaking, require tests for quality monitoring during their production processes. In these days, however, a microbial production process is a microbiological black box. That is, microbes like enhancers, spoilers and pathogens are neither enumerated nor specified during the monitoring process. This is true for the entire process or each individual process step within that process. Instead, physical tests, such as a pH test, a Titratable Acid test, a Volatile Acid test, a Malic Acid test, a Free SO₂ test and an Alcohol content test, are selectively performed, according to the purposes to be served for manufacturing process, only to provide indirect indications of microbial activities. These physical tests are “surrogate” microbiological tests and cannot provide direct, real-time indications of microbial activities during the microbial production processes.

Heterotrophic plate count (HPC) is a microbiological test performed today to provide an indication of microbial activities, but HPC only counts media-dependent microbes. Besides, HPC is performed by counting colony-forming units (CFUs) growing on agar that has been incubated for long periods of time (up to 8 days). Since this test can only indicate microbiological activity in the sample at the time the sample was taken, this test is primarily used for quality control and only at the very end of the process.

EP1688740A1 teaches testing a sample taken from a winemaking process, using the flowcytometry (FCM) to measure cell viabilities. However, this patent publication is totally silent about providing comprehensive test results on samples taken throughout the winemaking process.

### SUMMARY

The present invention is to provide rapid microbiological test reports, using microbiological testing devices, such as fluorescent flow cytometry (FCM), fluorescent microscopy, and polymerase chain reaction (PCR), HPC, etc., to create a true and comprehensive microbiological description of each step of microbial production process in real-time. These microbiological descriptions are combined to create a baseline microbiological description or “Fingerprint” of the entire process. This type of description has never been used or even considered in the past.

Among the above listed microbiological test methods, the FCM test is a representative microbiological test method for the present invention and can provide counts of microbes in a sample in near real-time. The present invention contemplates counting microbes in samples collected at multiple process steps throughout a microbial production process, such as a winemaking process, a beer making process, a spirit making process and a wastewater treatment process. The microbial test results are grouped to prepare a comprehensive microbiological test results, called a “Fingerprint.” Thus, a Fingerprint packages test results of multiple microbiological tests conducted throughout the microbial production process. The present invention will be described using exemplary use cases in which the FCM test is a representative microbiological test method, and winemaking is a representative microbial production process. However, descriptions of the present invention should not be restrictively construed to limit test methods to the FCM test and should not be restrictively construed to limit its applications to winemaking. There is nothing in the nature of the invention that limits test methods to a particular test method or limits applications of the present invention to a particular microbial production process.

The FCM test can be used to quantify the specific microbe species. A reagent used in the FCM test may contain a gene probe conjugated with a fluorescent dye. The gene probe functions as an antigen to recognize a specific microbe species in a sample. The reagent is mixed with samples collected throughout a microbial production process. If a viable microbe corresponding to the gene probe exists in the sample, the gene probe reacts and attaches to the viable microbe. The sample mixed with the reagent is measured by an FCM analyzer to count the gene probes attached to the specific microbe species. The FCM analyzer senses optical signals caused by the fluorescent dye attached to the microbe via the gene probe. The FCM analyzer counts the viable microbes in the samples based on the optical signals. The same test can be performed by F. Microscopy; however, its test morphology can be observed but enumeration of the microbes is not possible.

The FCM test can also be used to quantify total viable and non-viable microbial activity in a sample. Instead of the gene probe, the reagent may contain a fluorescent composition designed to react with any microbe species. The sample mixed with the fluorescent reagent is measured by the FCM analyzer, which senses optical signals caused by the fluorescent composition. The FCM analyzer counts the microbes in the sample based on the optical signals.

The Fingerprint prepared according to the present invention is reported to the entity employing a microbial production process. In the winemaking process, for example, microbiomes play an important role and may have an effect on the quality of wine products. It is therefore important to monitor the population of the microbiomes active in the winemaking process in order to control the winemaking processes. For example, some microbes may work to enhance the winemaking process. An exemplary enhancer for the winemaking process is yeast. Yeasts utilize glucose and fructose, the principal sugars in grape juice, and metabolize them via the Embden-Meyerhof-Parnas (glycolytic) pathway, to pyruvate. This pathway furnishes the yeast cells with energy and with reducing power, for cellular biosyntheses. Under anaerobic conditions, the yeasts decarboxylate pyruvate, in a reaction catalyzed by pyruvate decarboxylase, to yield acetaldehyde and CO₂. The final step in alcoholic fermentation is catalyzed by alcohol dehydrogenase and involves the reduced coenzyme NADH, and results in the reduction of acetaldehyde to ethanol.

The population of the enhancers during the winemaking process is counted and monitored by multiple FCM tests according to the present invention that is performed during the winemaking process. Normally, winemakers will measure the amount of sugar being consumed during the fermentation process to create a rough estimate of efficacy of the yeast cells present in their fermentation vessel. The problem with this surrogate form of measurement is that the fermentation vessel does not contain a single yeast microbe, but rather it contains a biocenosis or ecosystem of many microbes where yeast is the dominant species. In a biocenosis, the community of microbes will try to compensate for the die-off of the dominant species by having fewer cells work harder to achieve the same result. Therefore, if the only measurement taken measures the efficacy of the biocenosis, it is impossible to detect when your dominant species is starting to die off. In this case, it is only possible to detect a problem when the entire biocenosis is so overloaded that it ceases to function. At that point, it is too late to correct the winemaking process because the population of the dominant species is almost zero, and the process is left with a stuck or stopped fermentation. By using the rapid FCM test to enumerate the yeast population inside the fermentation vessel, it becomes possible to monitor the number of yeast cells and detect when there is a negative trend in the population which gives the winemaker an early warning so the winemaker has time to take action to mitigate the problem before the winemaker experiences a sluggish, stuck or stopped fermentation.

The graph set forth below shows how FCM can be used to predict die-out of enhancers.

By monitoring the population of the enhancers using the present invention, its die-out time can be predicted. In the above graph, at the outset, the enhancers maintain a high population, and the efficiency of the enhancer is high. If the environment changes, and because of that, the population of the enhancers decreases, the change in population can be detected by FCM and a remedy can be employed to prevent die-off. It is imperative for winemakers to be able to measure when the enhancers are starting to die so that the winemakers can timely make necessary adjustments to the winemaking process. The Fingerprints of the present invention provide a baseline under optimal conditions so that the winemakers surveil their process and take necessary action when an initial sign of anomalous results is detected.

Some microbes are called spoilers and detrimental to winemaking. For example, Lactic acid bacteria, or LAB, are found on grape surfaces and in grape must during wine fermentations and include both lactobacilli (e.g., Lactobacillus brevis) and lactococci (e.g., Oenococcus oeni). LAB may cause detrimental aspects to wine sensory attributes. Acetic acid bacteria are always present from the grape to the finished wine product. They need oxygen or high redox potential for growth, and their deleterious activity (oxidation of ethanol to acetic acid) is prevented by the low redox of the medium during the fermentation. During ageing, wine is therefore protected from aeration. The non-Saccharomyces yeast of the species Brettanomyces bruxellensis is the most off-flavor producing microorganism redoubtable in red winemaking. By producing ethyl phenols, it causes considerable loss. It is an increasing concern for winemakers, but early detection is now possible using specific molecular-based diagnostic methods (i.e., the PCR method and the FCM method). Some strains of lactic acid bacteria, even in the O. oeni species, can produce biogenic amines from amino acids. They are considered as spoiling strains because biogenic amines may have undesirable effects for some wine products. Other strains may produce ropiness. All these specific strains are detectable by specific PCR analysis.

The populations of the spoilers may be counted and monitored by multiple FCM tests according to the present invention. Via the monitoring using the present invention, a beginning of exponential growth of the spoiler may be detected. The graph set forth below shows an exponential increase in the spoiler populations measurable by the present invention.

As shown in the above graph, at the outset, the population of spoilers is low but grows exponentially when the time passes at a certain point. The Fingerprints of the present invention provide a sign of the beginning of exponential growth of the spoiler population so that the winemakers can take measures, such as sterilization, to suppress the growth of spoiler population before the wine products become completely spoiled.

A balance between the enhancers and the spoilers can be monitored using the PCM methods according to the present invention. The graph set forth below shows both a sign of decrease in the enhancer population and a sign of increase in spoiler population.

As shown above, the Fingerprints of the present invention tell the winemakers when to perform sterilization and when to add enhancers before the enhancer dies out and before the population of spoilers grow exponentially.

Another application of the present invention to the winemaking process is to make sure that no microbes are present in the finished bottled wine products.

Specifically, the present invention provides a method for preparing a quality assurance and quality control report on microbial process. The method comprises receiving multiple sets of microbial test results and test attributes related to the multiple sets of microbial test results, wherein the one set, among the multiple sets, of microbial test results is obtained from at least one testing device that each performs a different microbial testing method on samples collected at process points of interest in a microbial process. The method further comprises grouping the multiple sets of microbial test results to prepare fingerprints that each represent a different group of microbial test results, wherein the test attributes include: key information that identifies a fingerprint; a manufacturing line ID that identifies the microbial process; a process ID that identifies a purpose of each microbial test; and a test property that identifies a type of each test. The method further comprises, in association with the key information of a particular fingerprint, storing the particular fingerprint in a fingerprint library created in a database. The method further comprises, in response to a request to retrieve the particular fingerprint stored in a database that contains other fingerprints, searching the fingerprint library in the database for the particular fingerprint using the key information contained in the request.

In an alternative aspect of the present invention, grouping the multiple sets of microbial test results may comprise grouping the multiple sets of microbial test results according to batches of products produced by the microbial process, wherein the key information comprises different key information for each of the batches of the products.

In an alternative aspect of the present invention, grouping the multiple sets of microbial test results may comprise grouping two sets of microbial test results differently where the two sets of microbial test results are obtained from two different microbial processes, respectively, wherein the key information comprises different key information for the two sets of microbial test results.

In an alternative aspect of the present invention, grouping the multiple sets of microbial test results may comprise grouping the multiple sets of microbial test results according to lots of products produced by the microbial process, wherein the key information comprises different key information for each of the lots of the products.

In the present invention, the fingerprint may uniquely identify a product produced by the microbial process with at least one of (i) a batch number of the product, (ii) a lot number of the product or (iii) a production year of the product.

In the present invention, the fingerprint may include chronological information representative of a timeline of a plurality of tests performed at different points in time during a single step of interest in the microbial process.

In the present invention, the manufacturing line ID may include at least one of (i) an industry standard description related to the microbial process, (ii) a process location where the microbial process is performed, or (iii) a facility where the microbial process is performed.

In the present invention, the fingerprint may be associated with auxiliary information that includes at least one of (i) a quality of a product produced by the microbial process, (ii) conditions under which the microbial process is performed or (iii) a process adjustment that includes at least one of a process control, a process optimization or a troubleshooting that is taken during the microbial process.

In the present invention, the multiple sets of microbial test results may include results of tests performed in the microbial process for producing one of a wine product, a beer product or a spirit product.

In the present invention, the multiple sets of microbial test results may include results of tests performed in the microbial process for wastewater treatment.

The present invention may be implemented in a computer system for preparing a quality assurance and quality control report on microbial process. The computer system is programed to perform a microbial process that includes receiving multiple sets of microbial test results and test attributes related to the multiple sets of microbial test results, wherein the one set, among the multiple sets, of microbial test results is obtained from at least one testing device that each performs a different microbial testing method on samples collected at process points of interest in a microbial process. The microbial process further include grouping the multiple sets of microbial test results to prepare fingerprints that each represent a different group of microbial test results, wherein the test attributes include: key information that identifies a fingerprint; a manufacturing line ID that identifies the microbial process; a process ID that identifies a purpose of each microbial test; and a test property that identifies a type of each test. The microbial process further include, in association with the key information of a particular fingerprint, storing the particular fingerprint in a fingerprint library created in a database. The microbial process further includes, in response to a request to retrieve the particular fingerprint stored in a database that contains other fingerprints, searching the fingerprint library in the database for the particular fingerprint using the key information contained in the request.

In the microbial process performed by the computer system according to the present invention, grouping the multiple sets of microbial test results may comprise grouping the multiple sets of microbial test results according to batches of products produced by the microbial process, wherein the key information comprises different key information for each of the batches of the products.

In the microbial process performed by the computer system according to the present invention, grouping the multiple sets of microbial test results may comprise grouping two sets of microbial test results differently where the two sets of microbial test results are obtained from two different microbial processes, respectively, wherein the key information comprises different key information for the two sets of microbial test results.

In the microbial process performed by the computer system according to the present invention, grouping the multiple sets of microbial test results may comprise grouping the multiple sets of microbial test results according to lots of products produced by the microbial process, wherein the key information comprises different key information for each of the lots of the products.

In the microbial process performed by the computer system according to the present invention, the fingerprint may uniquely identify a product produced by the microbial process with at least one of (i) a batch number of the product, (ii) a lot number of the product or (iii) a production year of the product.

In the microbial process performed by the computer system according to the present invention, the fingerprint may include chronological information representative of a timeline of a plurality of tests performed at different points in time during a single step of interest in the microbial process.

In the microbial process performed by the computer system according to the present invention, the manufacturing line ID includes at least one of (i) an industry standard description related to the microbial process, (ii) a process location where the microbial process is performed, or (iii) a facility where the microbial process is performed.

In the microbial process performed by the computer system according to the present invention, the fingerprint is associated with auxiliary information that includes at least one of (i) a quality of a product produced by the microbial process, (ii) conditions under which the microbial process is performed or (iii) a process adjustment that includes at least one of a process control, a process optimization or a troubleshooting that is taken during the microbial process.

In the microbial process performed by the computer system according to the present invention, the multiple sets of microbial test results include results of tests performed in the microbial process for producing one of a wine product, a beer product or a spirit product.

In the microbial process performed by the computer system according to the present invention, the multiple sets of microbial test results include results of tests performed in the microbial process for wastewater treatment.

### BRIEF DESCRIPTION OF THE DRAWINGS

FIG. 1 is an exemplary diagram showing application of the present invention to a winemaking process;

FIG. 2 is a functional representation of an exemplary computer system for implementing a first embodiment of the present invention;

FIG. 3 shows an exemplary data structure of a fingerprint;

FIG. 4 is a functional representation of an exemplary computer system for implementing a second embodiment of the present invention;

FIG. 5 is a block diagram showing a schematic representation of a winemaking process;

FIG. 6 shows an exemplary structure of the Fingerprint obtained by a computer system shown in FIG. 5;

FIG. 7 shows another example of data structure of the fingerprint;

FIG. 8 is a block diagram according to a third embodiment of the present invention showing a beer making process;

FIG. 9 is a data structure of a fingerprint obtained from the beer making process shown in FIG. 8;

FIG. 10 a block diagram according to a fourth embodiment of the present invention showing a sprit making process;

FIG. 11 a data structure of a fingerprint obtained from the spirit making process shown in FIG. 10;

FIG. 12 is a block diagram according to a fifth embodiment of the present invention showing a wastewater treatment process;

FIG. 13 shows a data structure of a fingerprint obtained from the wastewater treatment process shown in FIG. 12;

FIG. 14 is a block diagram according to a sixth embodiment of the present invention showing a wastewater treatment process using the activated sludge process; and

FIG. 15 a data structure of a fingerprint obtained from the wastewater treatment process shown in FIG. 14.

### DETAILED DESCRIPTION

In the following description of embodiments of the present invention, various examples will be discussed. For the purpose of explanation, specific configurations and details are set forth in order to provide a thorough understanding of the examples and the present invention. However, it will also be apparent to one skilled in the art that the details provided to explain the examples are not restrictive, and the examples may be practiced without the specific details discussed below. Furthermore, in the following descriptions, well-known features may be omitted or simplified in order not to obscure the examples being described.

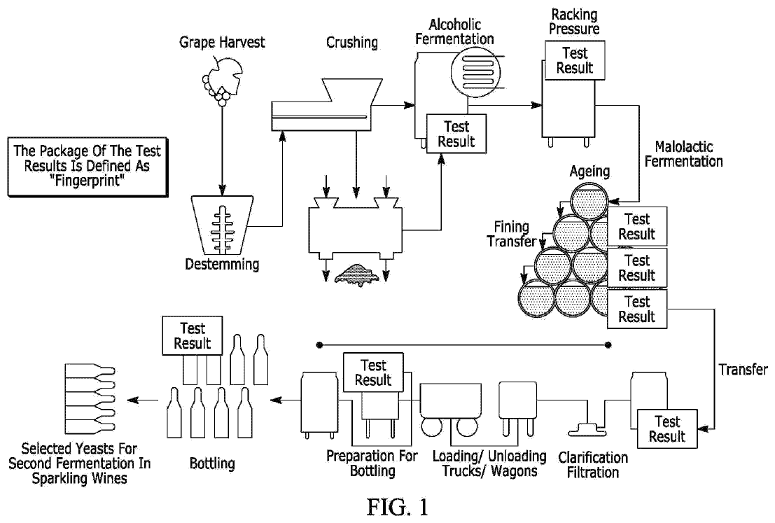

FIG. 1 is an exemplary diagram showing the application of the present invention to the winemaking process. As shown in FIG. 1, the winemaking process includes steps of grape harvest, crushing of harvested grapes, alcoholic fermentation, packing pressure, malolactic fermentation (optional), ageing, clarification filtration, loading/unloading tracks/wagons, preparation for bottling and bottling. As shown in FIG. 1, the present invention performs multiple microbiological tests at steps of interest throughout the winemaking process. In FIG. 1, the first step of interest at which the microbiological test according to the present invention is performed is the alcoholic fermentation step. The microbiological test according to the present invention is also performed at the racking pressure step, the ageing step, the transfer step, the bottling preparation step and the bottling step. The microbiological test according to the present invention is performed three times during the ageing step at three different timings. The microbiological test according to the present invention includes an analysis of activities of microbes at each of the above identified steps of interest in the winemaking process. The test results can present a number of microbes in the microbiomes and can identify the species of microbes detected at the respective steps of interest in the winemaking process. The test results are then grouped according to a prescribed specification. In the present invention, a set of grouped microbiological test results is referred to as a “Fingerprint.”

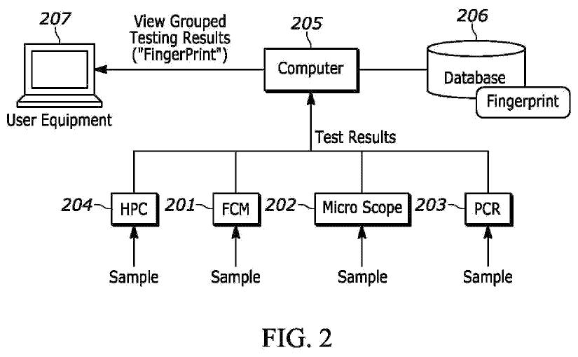

A Fingerprint is a collection of microbial analysis data collected from testing devices, such as a flow cytometer (FCM) analyzer, a testing device that performs a fluorescent microscopic analysis, a polymerase chain reaction (PCR) analyzer and a device that performs a heterotrophic plate count (HPC) analysis. FIG. 2 is a functional representation of an exemplary computer system for implementing a first embodiment of the present invention. In FIG. 2, a sample obtained from each of above-identified steps of interest in the winemaking process is provided to each of a FCM analyzer 201, a fluorescent microscopic analysis device 202, a PCR analyzer 203 and a HPC analysis device 204, where the provided sample is analyzed by these testing devices according to the specific analyzing functionality of the respective testing devices. A sample may be provided to only some of the testing devices 201-204 depending on the purpose of analysis and depending on the kind of microbial product to be produced.

The FCM analyzer 201 can enumerate all viable and non-viable microbes, as well as delineate sub-populations of microbiome species contained in a sample, based on differences in cell size, morphology, and differences in detected fluorescence. In the specific application of the FCM analyzer 201 to monitoring the winemaking process, fluorescence tagging can be applied to monitor activities of a microorganism of interest, such as Brettanomyces yeast.

In the specific application of the fluorescent microscopic analysis device 202 to monitoring the winemaking process, the fluorescent microscope analysis device can be equipped with a camera and a computerized image analysis module that can identify and classify microorganisms and their species of interest, based on microscopic images of the microorganisms.

The PCR analyzer 203 can rapidly produce and amplify millions to billions of copies of a specific segment of DNA, which can then be studied in greater detail. In the specific application of the PCR analyzer 203 to monitoring the winemaking process, the PCR analyzer 203 is used to detect specific strains of microorganisms that are beneficial or detrimental to the winemaking process.

Heterotrophic plate count (HPC) is a method that measures colony formation units on culture media of heterotrophic microorganisms in the winemaking process. Thus, in the specific application of the HPC analysis device 204 to monitoring the winemaking process, HPC analysis device 204 can be used to measure the overall microbial quality in the winemaking process.

Returning to FIG. 2, a sample obtained from each of above-identified steps of interest in the winemaking process is analyzed by each of the FCM analyzer 201, the fluorescent microscopic analysis device 202, the PCR analyzer 203 and the HPC analysis device 204, which provides its microbial test results to the computer 205. It should be noted that although FIG. 2 illustrates that each of the testing devices 201-204 performs its analysis on the sample and provides test results to the computer 205, at least one of the testing devices 201-204 may be provided with a sample for analysis, depending on microbes of interest to monitor at each of the above identified steps (see FIG. 1) of interest in the winemaking process.

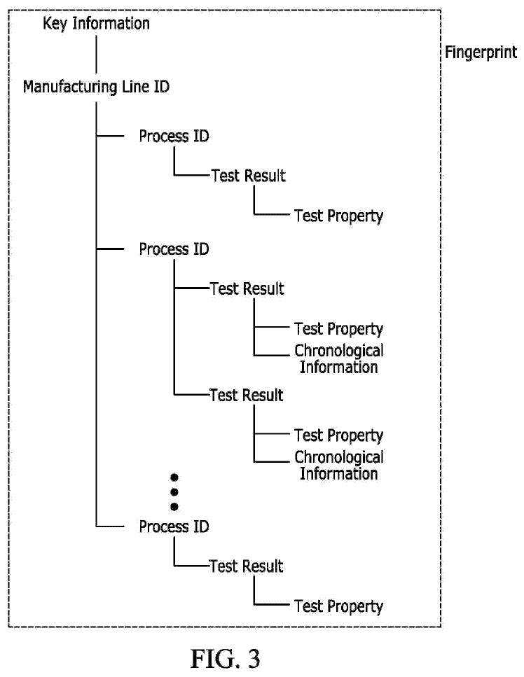

The computer 205 receives microbial test results from the test devices 201-204 and groups the received test results according to any specification the user creates. The computer 205 then stores each of the grouped test results on a database 206 as a Fingerprint. Through a terminal 207, which may be a mobile terminal, a PC, etc., a user can view Fingerprints stored in the database 206. FIG. 3 shows an exemplary data structure of the Fingerprint.

### “Key Info”

“Key info” or key information provides an identification of the Fingerprint. The key information may be an identifier including information for uniquely identifying the Fingerprint per a batch, for example, of wine products produced from the winemaking process. The identifier may, for example, represent a lot number and a production Year/Month indication associated with a batch of the wine products. The Key information may be an identifier for uniquely identifying different Fingerprints, each of which is obtained from a different winemaking process. The Key information may also be an identifier for uniquely identifying different Fingerprints, each of which is obtained from the same winemaking process under different production conditions. Multiple wine products produced by the same winemaking process may be assigned with the same winemaking process ID. A winemaking process may be repeated to produce different wine products over different production cycles (e.g., monthly, yearly). The Key information may include an identification for a batch of wine products tagged with production vintage information in which the batch of wine products is produced. Accordingly, the microbial test results may be grouped according to a batch of wine products, or a vintage of wine products. For example, the Key information may include an identification of a batch (a) of wine products that identifies its lot number as “lot A” and its vintage as “vintage A.” As to a batch (b) of wine products, the Key information includes an identifier identifying its lot number as “lot B” and its vintage as “vintage B.” Accordingly, the Fingerprint uniquely identifies wine products with a combination of (i) a batch number, (ii) a lot number and (iii) a vintage and thus makes possible to provide quality assurance for respective subsets of a particular batch of wine products.

### “Manufacturing Line ID”

“Manufacturing line ID” identifies a winemaking process by at least one of (i) an industry standard description, (ii) a manufacturing location, or (iii) a facility of winemaking. The Manufacturing line ID may contain a text, such as “Wine/Burgundy/XYZ Winery/Santa Rosa/CA/Facility 1.”

### “Process ID”

“Process ID” identifies a microbial test performed in the winemaking process for quality assurance or quality control in association with a manufacturing line ID. Examples of process ID may be an alphabetic/numeric identification, e.g., “QA1” and a text identification, e.g., “Fermentation” or “Malolactic Fermentation.”

### “Test Result”

“Test result” includes results of microbial tests performed by the test methods 201-204. The test results may contain quantitative and qualitative analyses of a sample and descriptive information describing the analysis results. Examples of test result may be X Cells/ml, Y CFU (Colony Forming Units), Scattergram plot, Positive/Negative, Percentage, Microscopy image, for example.

### “Test Property”

“Test property” contains information regarding the properties of microbiological test performed, e.g., OenoCount (FCM), GramCount (FCM), Lactic Acid Bacteria (F. Microscopy), BrettCount (FCM), Saccharomyces (PCR), and Saccharomyces (HPC). The test property may include information regarding a date on which the test is performed.

### “Chronological Info.”

When a plurality of microbial tests are performed at different points in time during a single step of interest, the chronological information may contain information on a timeline of the plurality of tests. Examples of chronological information may be ordinal number information, such as “#1”, “#2”, . . . . The ordinal number may be added with other information, such as “Process ID” (e.g., “QA1.1”, “QA1.2”, . . . ). The chronological information may be substituted by information regarding a date information included in the test property. The chronological information will be discussed in more detail later.

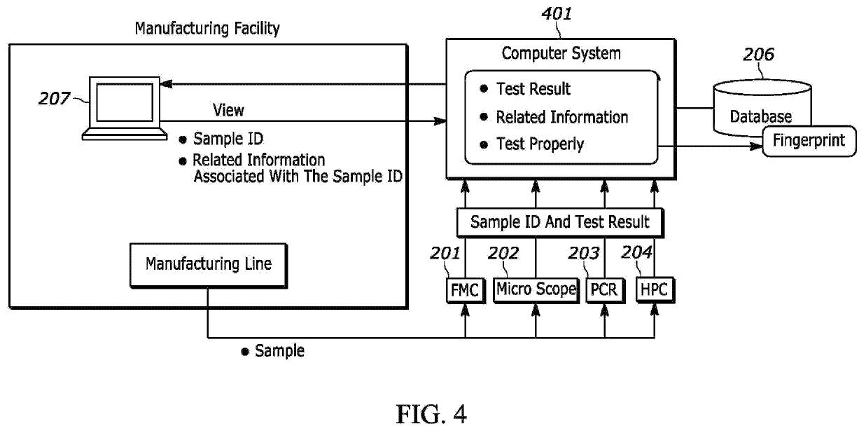

FIG. 4 is a functional representation of an exemplary computer system for implementing a second embodiment of the present invention. A sample taken at each of the above-identified steps of interest in the winemaking process is shipped from the manufacturing facility to a testing site where the samples are analyzed by at least one of the FCM analyzer 201, fluorescent microscopic analysis device 202, the PCR analyzer 203 or HPC analysis device 204. Each sample for testing site is stored in an individual container to which a computer readable sample ID, such as a bar-code or a QR code, is attached. The computer readable sample ID is generated by the winemaker and identifies sample attributes that include the Key Info, such as the Manufacturing line ID, the Process ID, the Test property and the Chronological Info. Alternatively, the winemaker may provide the sample attributes to the computer system 401, which returns a computer readable sample ID for printing by the winemaker. The winemaker prints out the computer readable sample IDs and attach them to individual sample containers.

The samples may be tested at a testing facility owned by the winemaker. The samples may alternatively be tested at a testing facility operated by a testing company hired by the winemaker. Testing at the facility of the testing company requires transportation of the samples from the winemaking facility and may affect the real-time nature of the requirement for obtaining test results, while test results from a testing company are in general more reliable.

In FIG. 4, a sample ID that identifies the sample attributes of a tested sample may be provided to a computer system 401, along with microbial test results from the test methods 201-204. The computer system 401 processes the microbial test results, the sample ID by associating the test results with the sample ID. The computer system 401 groups the received test results according to the Key information in the sample ID. Sets of grouped test results are stored in the database 206 as Fingerprints of the winemaking process.

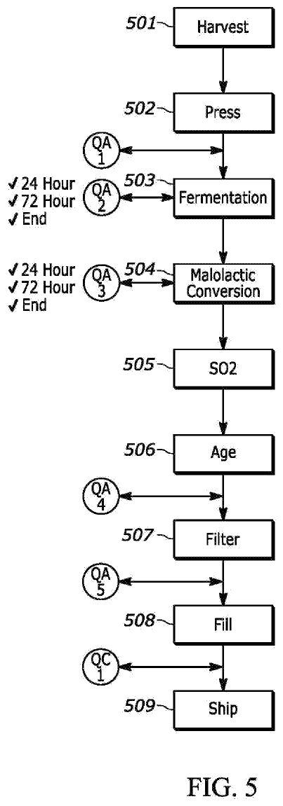

Moving on to FIG. 5, the data structure of the Fingerprint will further be explained. FIG. 5 is a block diagram showing a schematic representation of a winemaking process. As shown in FIG. 5, the winemaking process starts with a step of “Harvest” 501, in which grapes are harvested. The harvest step 501 is followed by a step of “Press” 502, in which harvested grapes are crushed and pressed. The process step 502 is followed by a step of “Fermentation” 503, in which alcoholic fermentation takes place. The fermentation step 503 is followed by a step of “Malolactic Fermentation” 504, in which malic acid is converted to lactic acid to reduce acidity and enhance aroma and flavor. The malolactic fermentation step 504 is followed by a step of “SO₂” 505, in which sulfur dioxide is added as a preservative to prevent oxidation and microbial spoilage. The SO₂ step 505 is followed by a step of “Age” 506, in which the wine product is aged. The ageing step 506 is followed by a step of “Filter” 507, in which the aged wine product is filtered to remove wine sludges and residues. The filter step 507 is followed by a step of “Fill” 508 and a step of “Ship” 509, which wine products are bottled and shipped.

As shown in FIG. 5, a sample is collected between the process step 502 and the fermentation step 503. This sample collection is referred to in FIG. 5 as QA1. “QA” is an indication of a process ID that means “quality assurance.” QA “1” is an indication of a chronological order of the test that means the first quality assurance test in the winemaking process. Another sample is collected during the fermentation step 503 (QA2). Process QA2 is performed three times at the end of 24 hours, the end of 72 hours and the end of the process 503. Another sample is collected during the malolactic fermentation step 504 (QA3). Process QA3 is performed three times at the end of 24 hours, the end of 72 hours and the end of the step 504. Another sample is collected between the age step 506 and the filter step 507 (QA4). Another sample is collected between the filter step 507 and the fill step 508 (QA5). A last sample is collected between the fill step 508 and the ship step 509. Returning to FIG. 4, these collected samples are analyzed by the test methods 201-204. The microbial test results from the test methods 201-204 are grouped by the computer system 401 to form a Fingerprint of the winemaking process.

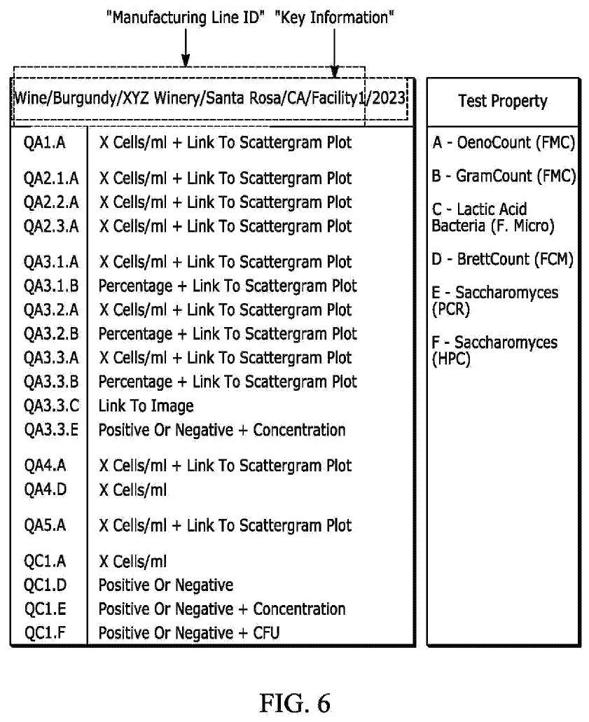

FIG. 6 shows an exemplary structure of the Fingerprint obtained by the computer system 401 shown in FIG. 5. In FIG. 6, the Key information includes a string of texts “Wine/Burgundy/XYZ Winery/Santa Rosa/CA/Facility 1/2023,” which represents an identification of the winemaking process by an original region of wine, a name and a location of winery and a wine production date. A string of texts “Wine/Burgundy/XYZ Winery/Santa Rosa/CA/Facility” represents a manufacturing line ID.

The Fingerprint shown in FIG. 6 contains chronological information and a test property of microbial tests performed. For example, “QA2.1.A”, “QA2.2.A” and “QA2.3.A” represent a chronological order of the performed tests and a test property. As explained above, “QA2” represents a process ID that means the second quality assurance test performed in the winemaking process. Further, in QA2.“1.”A, QA2.“2.” A and QA2.“3.”A, numbers 1, 2 and 3 represent ordinal numbers according to which three tests are chronologically performed during the fermentation step 503 at the end of 24 hours, the end of 72 hours and the end of the step 503 as shown in FIG. 5. “A” represent a test property that means an OenoCount by the FCM analyzer 201. Examples of the test property are explained below:
A—OenoCount (FCM): an enumeration of viable microorganisms (bacteria and yeast)
B—GramCount (FCM): an enumeration of viable Gram Positive and Gram Negative microorganisms
C—Lactic Acid Bacterial (F. Micro): an image showing highlighted Lactic Acid Bacteria
D—BrettCount (FCM): an enumeration of viable Brettanomyces present
E—Saccharomyces (PCR): a digital test result showing the presence of Saccharomyces which can also contain an estimated concentration
F—Saccharomyces (HPC): the number of colony forming units (CFUs)

A text string “X Cells/ml” represents a number of viable cells (X) per milliliter in the sample that is measured by the FCM analyzer 201. A text string “Scattergram Plot” represents a 2-dimensional or 3-dimensional graph created by the FCM analyzer 201 that shows clusters of detected microorganisms. “Percentage” represents a percentage of certain cells among the cells in the sample detected by the FCM analyzer 201. “Concentration” represents a concentration of certain cells detected by the FCM analyzer 201. “Positive” or “Negative” represents detection or non-detection of certain cell species measured by the FCM analyzer 201, a fluorescent microscopic analysis device 202, the PCR analyzer 203, or the HPC analysis device 204.

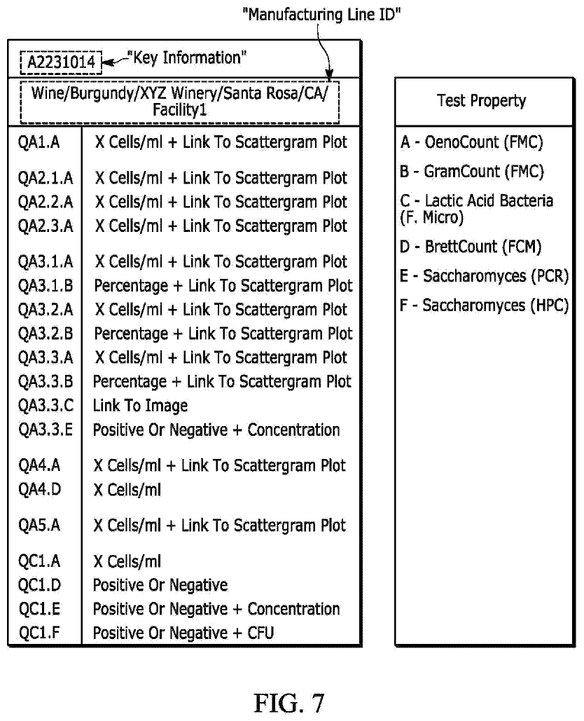

FIG. 7 shows another example of data structure of the Fingerprint. In FIG. 7, the Key information includes “A2231014,” which uniquely identifies a batch of wine products produced in the winemaking process identified by the manufacturing line ID.

The Fingerprint may be associated with auxiliary information that includes, for example, at least one of (i) a quality of a product, (ii) conditions of each winemaking process performed or (iii) a process adjustment that includes at least one of a process control, a process optimization or a troubleshooting that is taken during the process. The auxiliary information is generated and coded by, for example, a winemaker and attached to sample containers. The computer system 401 may store a Fingerprint on the database 206 after the Fingerprint is associated with the above auxiliary information. When the Fingerprint is retrieved from the database 206, the auxiliary information may also be retrieved with the Fingerprint.

The Fingerprint associated with the auxiliary information makes it possible for the winemaker to repeat the winemaking process while maintaining the same quality of the wine products. For example, the auxiliary information may include (i) the quality of the product and (ii) the conditions (e.g., temperature, etc.) of each winemaking process. Then, the manufacturer can relate the microbial test results to the quality of the wine product and the conditions of the wine making process. For example, if the manufacturer finds the quality of the product with a particular vintage satisfactory, the manufacturer can attribute the satisfactory quality of the wine product to the conditions under which the winemaking process was performed. The microbial test results included in the Fingerprint are quantitative and qualitative representations of the successful conditions of the winemaking process. By adjusting the conditions during a new winemaking process to those for the successful winemaking process, the manufacturer can expect to duplicate wine products with similarly satisfactory quality.

The Fingerprint may be associated only with (i) the quality of the product and (iii) the process adjustment that is taken during the winemaking process, without (ii) conditions of each winemaking process performed. Then, the manufacturer can relate the quality of the product to the process adjustment that is taken during the winemaking process. For example, if the manufacturer finds the quality of a wine product with a particular lot of wine satisfactory, the manufacturer can attribute the satisfactory quality of the wine product to the process adjustment. The microbial test results included in the Fingerprint are quantitative and qualitative representations of the process adjustment. By making the same process adjustment during a new winemaking process, the manufacturer can expect to duplicate wine products with similarly satisfactory quality.

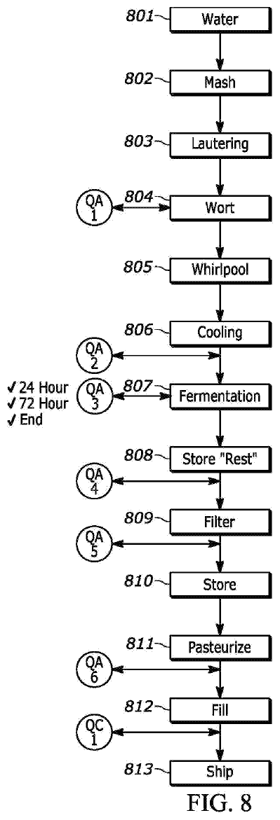

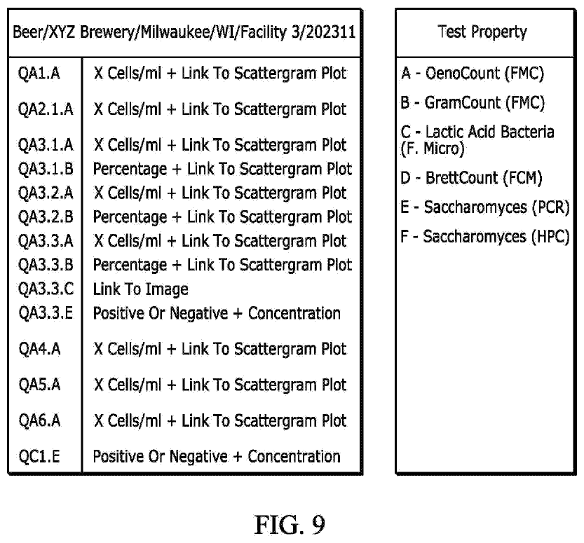

FIG. 8 is a block diagram according to a third embodiment of the present invention showing a beer making process. FIG. 9 is a data structure of a Fingerprint obtained from the beer making process shown in FIG. 8. As shown in FIG. 8, the beer making process begins with adding water to grain (Step 801). The grain added with water is mashed (Step 802), using enzymes to break down starches in the grain into sugars for fermentation. Lautering is then performed (Step 803) to separate sweet wort from the grain bed. Wort containing sugars is extracted (Step 804). In the next Whirlpooling step (Step 805), a circular current is created in the brew kettle. Then, the malt is cooled (Step 806) and fermented (Step 807). The fermented malt is then stored for resting (Step 808). After resting, raw beer is filtered out from the used grain (Step 809). The filtered raw beer is stored again for resting (Step 810). Then, the raw beer is pasteurized (Step 811) and bottled (Step 812) and shipped (Step 813). In the third embodiment of the present invention, a sample is collected during the Wort step 804 (QA1), then between the cooling step 806 and the fermentation step 807 (QA2), then during the fermentation step 807 (QA3), then between the store step 808 and the filter step 809 (QA4), then between the filter step 809 and the second store step 810 (QA5), then between the pasteurize step 811 and the fill step 812 (QA6) and lastly between the fill step 812 and the ship step 813 (QC1). The collected samples are tested by the test methods 201-204. In the third embodiment, a sample is collected three times during the fermentation step 807 at 24 hours, 3-5 days and the end of the step 807. QC1 is an indication of process ID for quality control that means the first quality control test.

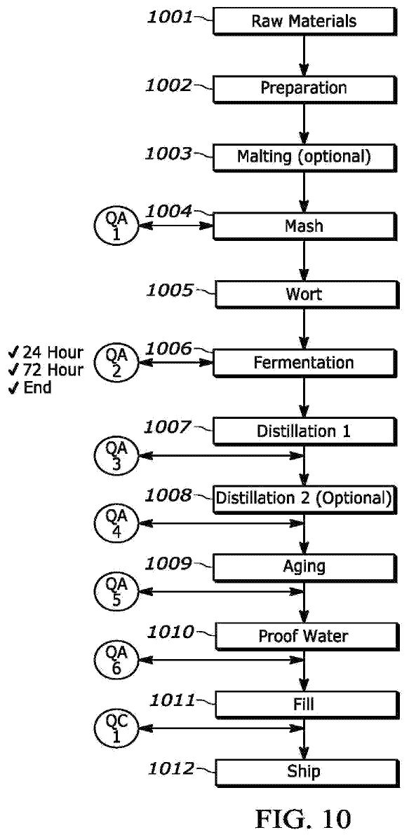

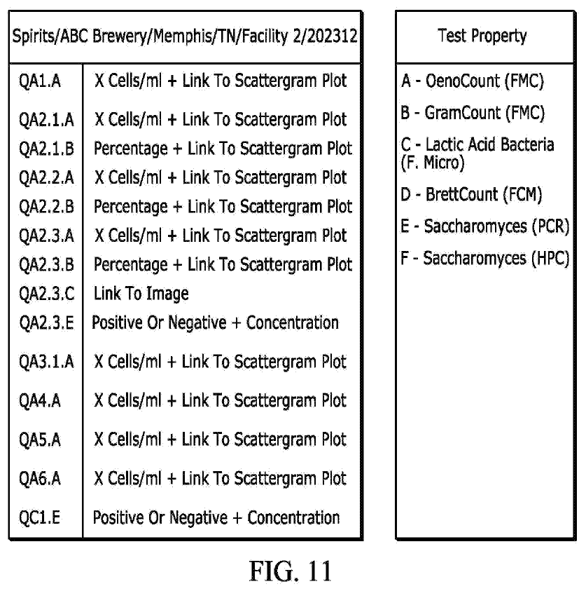

FIG. 10 a block diagram according to a fourth embodiment of the present invention showing a sprit making process. FIG. 11 shows a data structure of a Fingerprint obtained from the spirit making process shown in FIG. 10. The spirit making process begins with collecting raw materials (Step 1001). Raw materials differ depending on a kind of spirit to be produced. For example, the main cereal raw materials used by Scotch whisky producers are wheat and barley. Vodka is made from cereal grains (e.g., wheat, sorghum, or rye). The raw material is added with water for preparation (Step 1002). The raw material is malted (Step 1003) by steeping it in water and spreading it across a malting floor, allowing it to germinate. In the subsequent mashing step (Step 1004), the malted raw material is heated to induce natural enzymes (Amylase) to breakdown starch in the grain into fermentable sugars. Wort containing sugars is extracted (Step 1005). Then, the extracted wort is fermented (Step 1006). Alcohol is distilled (Step 1007). The distilled alcohol may go through second distillation (Step 1008). The distilled alcohol is aged (Step 1009). After ageing, the alcohol content is adjusted with water (Step 1010). The final product, spirit, is bottled (Step 1011) and shipped (Step 1012). In the fourth embodiment, a sample is collected for the first time during the mash step 1004 (QA1), for the second time during the fermentation step 1006 (QA2), for the third time between the first distillation step 1007 and the second distillation step 1008 (QA3), for the fourth time between the second distillation step 1008 and the aging step 1009 (QA4), for the fifth time between the aging step 1009 and the proof water step 1010 (QA5), for the sixth time between the proof water step 1010 and the fill step 1011 (QA6) and lastly between the fill step 1011 and the ship step 1012 (QC 1). In the fourth embodiment, a sample is collected three times during the fermentation step 1006 at the end of 24 hours, the end of 48 hours and the end of the step 1006. The collected samples are analyzed by the test methods 201-204. Microbial test results from the test methods 201-204 are provided to the computer system 401 to prepare a Fingerprint.

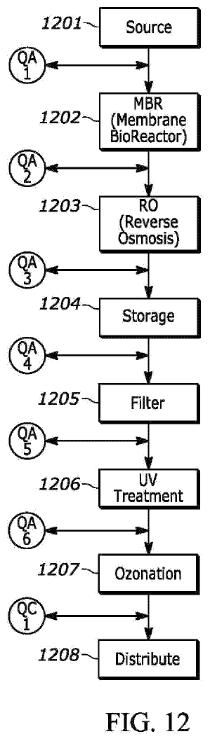

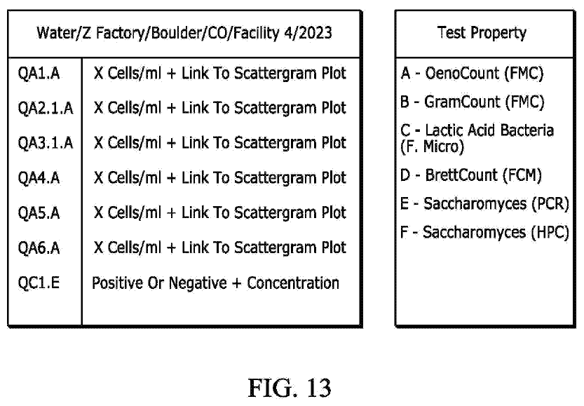

FIG. 12 is a block diagram according to a fifth embodiment of the present invention showing a direct potable recycle wastewater treatment process. FIG. 13 shows a data structure of a Fingerprint obtained from the wastewater treatment process shown in FIG. 12. The wastewater treatment process begins with introducing wastewater mixed liquor to be treated in a reservoir (S1201). A Membrane BioReactor (MBR) performs filtration in combination with a biological process that uses microbes to process wastewater (Step 1202). Reverse Osmosis (RO) is a process by which pressure forces wastewater through a permeable membrane to remove water contaminants such as lead, volatile organic compounds (VOCs), PFAS, arsenic, bacteria, and viruses (Step 1203). After Steps 1202 and 1203, water is stored (Step 1204) and filtered (Step 1205). Water then undergoes UV Treatment to remove emerging contaminants, such as pharmaceuticals and personal care products (Step 1206). Ozonation is a process where generated ozone is dissolved in water to kill microorganisms and remove organic and inorganic pollutants (Step 1207). Finally, purified water is distributed back for industrial and household use (Step 1208).

In the fifth embodiment, a sample is collected for the first time between the source step 1201 and the MBR step 1202 (QA1), for the second time between the MBR step 1202 and the RO step 1203 (QA2), for the third time between the RO step 1203 and the storage step 1204 (QA3), for the fourth time between the storage step 1204 and the filter step 1205 (QA4), for the fifth time between the filter step 1205 and the UV treatment step 1206 (QA5), for the sixth time between the UV treatment step 1206 and the ozonation step 1207 (QA6) and lastly between the ozonation step 1207 and the distribution step 1208 (QC1). The collected samples are analyzed by the test methods 201-204. The microbial test results from the test methods 201-204 are provided to the computer system 401 to prepare a Fingerprint.

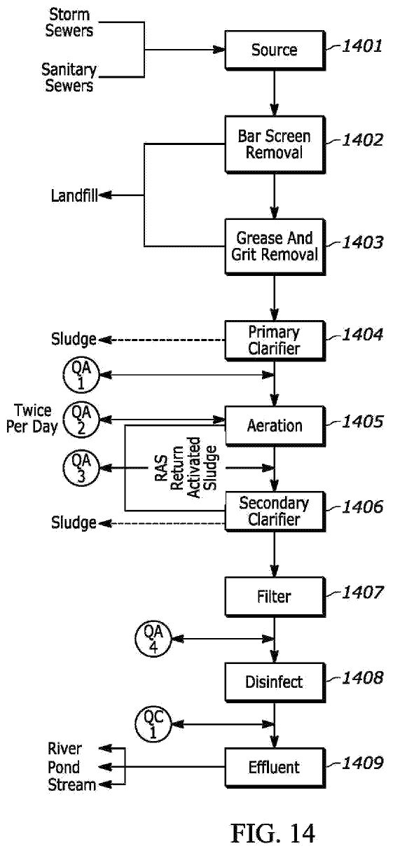

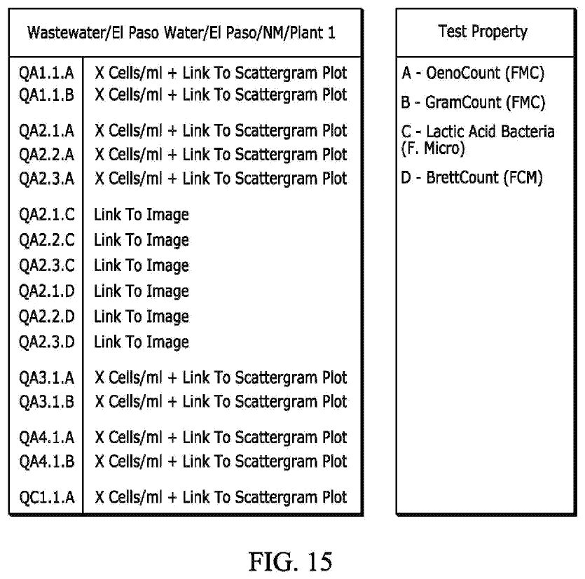

As a sixth embodiment of the present invention, the present invention can be applied to a wastewater treatment using the activated sludge process as shown in FIG. 14. FIG. 15 shows a data structure of a Fingerprint obtained from the wastewater treatment process shown in FIG. 14. The activated sludge process is a process that oxygen is added to sewage. The process is rich in naturally occurring oxygen and involves agitating sewage in an environment. The movement of sewage is sufficient to generate a rich oxygen mixture and there is no need to add oxygen artificially. In step 1401, wastewater is collected from storm sewers and sanitary sewers. In step 1402, the collected wastewater is filtered by a bar screen to remove landfill. Grease and grit are also removed from the collected wastewater in step 1403. Then, the collected wastewater after landfill is removed goes through a primary clarifier to remove sludges in step 1404. Then, air is added to the wastewater to allow aerobic biodegradation of the organic materials in the wastewater (step 1405). The wastewater then goes through a secondary clarifier in step 1406 to further remove sludges. Please note that activated sludge may be returned from the second clarifier step 1406 to the aeration step 1405. The wastewater then goes through a filter (step 1407) and is disinfected (step 1408). Finally, the treated wastewater is discharged into river, pond or water stream (step 1409).

In the sixth embodiment, a sample is collected immediately prior to the aeration step 1405 for the first time (QA1). A sample is collected for the second time at the aeration step 1405 (QA2). In the aeration step 1405, a sample is collected twice a day. A sample is also collected for the third time between the aeration step 1405 and the secondary clarifier step 1406 (QA3). A sample is also collected between the filter step 1407 and the disinfection step 1408 (QA4). A last sample is collected after the disinfection step 1408 before discharged into river, pond or water stream (QC1).

Returning to the winemaking process, winemakers may wish that microbial tests, according to the present invention, be performed on each product separately. For example, wine products, made from the same type of grapes, are distinguished by their vintages, or the years grapes are harvested, such as the year 2020, the year 2021, the year 2022 and the year 2023, or more importantly batches of wine products. There are multiple batches of wine products with an identical manufacturing line ID. A Fingerprint that includes an identifier of a batch can be distinguished from another Fingerprint that include an identifier identifying a different batch. Fingerprints with a batch identifier can be used to (i) troubleshoot the manufacturing line, (ii) optimize process(es) of the manufacturing line, (iii) act as a process template to duplicate the manufacturing process(es), (iv) act as a reference for process control. The Key information may include a batch identifier assigned to the same batch of winemaking process. Such batch identifier may be added to the Key information or may be included in the lot number information. Fingerprints representing different batch identifies with the same manufacturing line ID can provide a range of a microbial activity, e. g., the number of particular microbes, at each of the steps of interest. The winemaker adjusts the microbial activity at each of the steps of interest so that the detected microbial activity falls within the provided range. As a result, the winemaker can replicate a batch of wine product according to a past successful batch of wine product.

If a winemaker wishes a test provider to perform the microbial tests according to the present invention, the Key information may be provided by the winemaker to the test provider. When microbial test results are returned to the winemaker from the test provider, the Key information is also uploaded by the test provider to the computer system 401. The computer system 401, when preparing a Fingerprint for the returned test results, associates the Key information with the returned testing results. If the winemaker has the ability to perform the microbial test according to the present invention, the Key information is uploaded to the computer system 401 by the winemaker.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily occur to those skilled in the art, it is not desired to limit the invention to the exact construction and process described above. Accordingly, all suitable modifications and equivalents may be resorted to falling within the scope of the invention as defined by the claims that follow.

What is claimed is:

1. A method for preparing a quality assurance and quality control report on microbial process, the method comprising:
receiving multiple sets of microbial test results and test attributes related to the multiple sets of microbial test results, wherein the one set, among the multiple sets, of microbial test results is obtained from at least one testing device that each performs a different microbial testing method on samples collected at process points of interest in a microbial process;
grouping the multiple sets of microbial test results to prepare fingerprints that each represent a different group of microbial test results, wherein the test attributes include: key information that identifies a fingerprint; a manufacturing line ID that identifies the microbial process; a process ID that identifies a purpose of each microbial test; and a test property that identifies a type of each test;
in association with the key information of a particular fingerprint, storing the particular fingerprint in a fingerprint library created in a database; and
in response to a request to retrieve the particular fingerprint stored in a database that contains other fingerprints, searching the fingerprint library in the database for the particular fingerprint using the key information contained in the request.

2. The method according to claim 1, wherein grouping the multiple sets of microbial test results comprises grouping the multiple sets of microbial test results according to batches of products produced by the microbial process, wherein the key information comprises different key information for each of the batches of the products.

3. The method according to claim 1, wherein grouping the multiple sets of microbial test results comprises grouping two sets of microbial test results differently where the two sets of microbial test results are obtained from two different microbial processes, respectively, wherein the key information comprises different key information for the two sets of microbial test results.

4. The method according to claim 1, wherein grouping the multiple sets of microbial test results comprises grouping the multiple sets of microbial test results according to lots of products produced by the microbial process, wherein the key information comprises different key information for each of the lots of the products.

5. The method according to claim 1, wherein the fingerprint uniquely identifies a product produced by the microbial process with at least one of (i) a batch number of the product, (ii) a lot number of the product or (iii) a production year of the product.

6. The method according to claim 1, wherein the fingerprint includes chronological information representative of a timeline of a plurality of tests performed at different points in time during a single step of interest in the microbial process.

7. The method according to claim 1, wherein the manufacturing line ID includes at least one of (i) an industry standard description related to the microbial process, (ii) a process location where the microbial process is performed, or (iii) a facility where the microbial process is performed.

8. The method according to claim 1, wherein the fingerprint is associated with auxiliary information that includes at least one of (i) a quality of a product produced by the microbial process, (ii) conditions under which the microbial process is performed or (iii) a process adjustment that includes at least one of a process control, a process optimization or a troubleshooting that is taken during the microbial process.

9. The method according to claim 1, wherein the multiple sets of microbial test results include results of tests performed in the microbial process for producing one of a wine product, a beer product or a spirit product.

10. The method according to claim 1, wherein the multiple sets of microbial test results include results of tests performed in the microbial process for wastewater treatment.

11. A computer system for preparing a quality assurance and quality control report on microbial process, the computer system being programed to perform a microbial process that includes:
receiving multiple sets of microbial test results and test attributes related to the multiple sets of microbial test results, wherein the one set, among the multiple sets, of microbial test results is obtained from at least one testing device that each performs a different microbial testing method on samples collected at process points of interest in a microbial process;
grouping the multiple sets of microbial test results to prepare fingerprints that each represent a different group of microbial test results, wherein the test attributes include: key information that identifies a fingerprint; a manufacturing line ID that identifies the microbial process; a process ID that identifies a purpose of each microbial test; and a test property that identifies a type of each test;
in association with the key information of a particular fingerprint, storing the particular fingerprint in a fingerprint library created in a database; and
in response to a request to retrieve the particular fingerprint stored in a database that contains other fingerprints, searching the fingerprint library in the database for the particular fingerprint using the key information contained in the request.

12. The computer system according to claim 11, wherein grouping the multiple sets of microbial test results comprises grouping the multiple sets of microbial test results according to batches of products produced by the microbial process, wherein the key information comprises different key information for each of the batches of the products.

13. The method according to claim 11, wherein grouping the multiple sets of microbial test results comprises grouping two sets of microbial test results differently where the two sets of microbial test results are obtained from two different microbial processes, respectively, wherein the key information comprises different key information for the two sets of microbial test results.

14. The method according to claim 11, wherein grouping the multiple sets of microbial test results comprises grouping the multiple sets of microbial test results according to lots of products produced by the microbial process, wherein the key information comprises different key information for each of the lots of the products.

15. The method according to claim 11, wherein the fingerprint uniquely identifies a product produced by the microbial process with at least one of (i) a batch number of the product, (ii) a lot number of the product or (iii) a production year of the product.

16. The method according to claim 11, wherein the fingerprint includes chronological information representative of a timeline of a plurality of tests performed at different points in time during a single step of interest in the microbial process.

17. The method according to claim 11, wherein the manufacturing line ID includes at least one of (i) an industry standard description related to the microbial process, (ii) a process location where the microbial process is performed, or (iii) a facility where the microbial process is performed.

18. The method according to claim 11, wherein the fingerprint is associated with auxiliary information that includes at least one of (i) a quality of a product produced by the microbial process, (ii) conditions under which the microbial process is performed or (iii) a process adjustment that includes at least one of a process control, a process optimization or a troubleshooting that is taken during the microbial process.

19. The method according to claim 1, wherein the multiple sets of microbial test results include results of tests performed in the microbial process for producing one of a wine product, a beer product or a spirit product.

20. The method according to claim 1, wherein the multiple sets of microbial test results include results of tests performed in the microbial process for wastewater treatment.
